(19) United States
(12) Patent Application Publication
(10) Pub. No.: US 2009/0281476 A1
(43) Pub. Date: Nov. 12, 2009

(54) Method, Tool, and Kit for Making a Subcutaneous Pocket

(76) Inventors: David A. Loiterman, Oak Brook, IL (US); Michael G. Loiterman, Yorkville, IL (US)

Correspondence Address: VEDDER PRICE P.C., 222 N. LASALLE STREET, CHICAGO, IL, 60601, US

(21) Appl. No.: 12/463,074

(22) Filed: May 8, 2009

Related U.S. Application Data
(60) Provisional application No. 61/051,390, filed on May 8, 2008.

(51) Int. Cl. A61M 29/02; A61B 17/32; A61B 17/00

(52) U.S. Cl. 604/20; 604/96.01; 606/1

(57) ABSTRACT

The present disclosure generally relates to a tool, a kit including said tool, and a method of use of the tool or the kit for making a subcutaneous pocket in a layer of fat for the introduction of a subcutaneous medical device, and more specifically, a tool, kit, and method of use thereof using a calibrated inflatable volume with a guide to open a subcutaneous pocket at the appropriate location in a body by inflating the volume to the appropriate size. The tool is calibrated to rest against a layer of muscle using a guide wire and inflate in a specific shape to a specific volume to gently displace fatty tissue that may obstruct the implantation of a subcutaneous device between the skin and the ultimate resting place. The tools allows for creating localized lateral retraction in an area of interest without placing strain on the skin or the muscle. A kit using the tool further includes surgical equipment needed for the surgery.

### CROSS-REFERENCE TO RELATED APPLICATION

The present patent application claims priority from and the benefit of U.S. Provisional Patent Application No. 61/051,390, filed May 8, 2008, and entitled Method, Tool, and Kit for Making a Subcutaneous Pocket, which prior application is hereby incorporated herein by reference.

### FIELD OF THE DISCLOSURE

The present disclosure generally relates to a tool, a kit including said tool, and a method of use of the tool or the kit for making a subcutaneous pocket in a layer of fat for the introduction of a subcutaneous medical device, and more specifically, a tool, kit, and method of use thereof using a calibrated, inflatable volume with guide to open a subcutaneous pocket at the appropriate location in a body by inflating the volume to the appropriate size.

### BACKGROUND

Some devices, such as the device shown in U.S. Pat. No. 5,041,098, which describes a vascular access system for extra corporeal treatment of blood and is issued to one of the inventors of the current invention, must be implanted below the skin and made to rest against a layer of muscle or other hard tissue for their proper operation. FIGS. 3 and 4 shown how this sample device is installed between the skin and a layer of muscle fibers.

The human body stores fat between the human skin and a muscle layer. This fat is often not the proper resting place for some types of subcutaneous implanted devices such as the one shown in FIGS. 3 and 4. When small objects such as pellets are to be inserted, they do not require great volumes be displaced and can be inserted using either a puncturing tool as shown in FIG. 1 or a slider as shown in FIG. 2. For example, chips are inserted below the skin of the back of the neck of dogs for identification in case the animal runs away. These chips are the size of a grain of rice do not discomfort the animal. Thus, devices such as those shown in FIGS. 1-2 are appropriate.

In implanted devices such as the one shown in FIGS. 3 and 4, the surgeon must first make an incision in the skin of a desired size to allow introduction of the device, and fat must be removed or pushed aside while the device is positioned to rest against the muscle. For patients who are obese or morbidly obese, or for devices that must be inserted in locations where the human body generally stores more fat, this problem is compounded. What is needed is a tool or a method of surgical operation using a tool that allows for the easy installation of a subcutaneous implanted device in the proper location.

Also, during surgery, different team members of the surgery team are allowed to perform tasks of differing complexity. When one part of an overall procedure is simplified, that task can be assigned to be performed by a less experienced team member, thus saving costs for the overall surgery. Consequently, what is also needed is a kit and tool operating in tandem with a method of use of the kit or tool for preparing an implanted area that is easy to use and can be performed by less experienced surgeons.

### SUMMARY

The present disclosure generally relates to a tool, a kit including said tool, and a method of use of the tool or the kit for making a subcutaneous pocket in a layer of fat for the introduction of a subcutaneous medical device, and more specifically, a tool, kit, and method of use thereof using a calibrated inflatable volume with a guide to open a subcutaneous pocket at the appropriate location in a body by inflating the volume to the appropriate size. The tool is calibrated to rest against a layer of muscle using a wire guide and inflate in a specific shape to a specific volume to gently push aside fat cells that may obstruct the implantation of a subcutaneous device between the skin and the ultimate resting place. The tool allows for creation of localized lateral retraction in an area of interest without placing strain on the skin or the muscle. A kit using the tool further includes surgical equipment needed for the surgery.

### BRIEF DESCRIPTION OF THE DRAWINGS

Certain embodiments are shown in the drawings. However, it is understood that the present disclosure is not limited to the arrangements and instrumentality shown in the attached drawings.

FIG. 1 taken from the prior art illustrates a first known instrument for implanting sensors and solid materials in a subcutaneous fatty region.

FIG. 2 taken from the prior art illustrates a second known instrument for implanting articles in a subcutaneous fatty tissue region.

FIG. 3 is a front view of a patient with two possible subcutaneous implants positioned on the upper chest area according to an embodiment of the present disclosure.

FIG. 4 is a sectional view one possible implant as shown in FIG. 3 placed between a layer of muscle tissue and the skin according to an embodiment of the present disclosure.

FIG. 5 is diagrammatic representation of a possible embodiment of a tool for making a subcutaneous pocket for guiding and placing an implant of the present disclosure.

FIG. 6 is a sectional view according to cut line 6-6 as shown in FIG. 5 of the tool for making a subcutaneous pocket according to an embodiment of the present disclosure.

FIG. 7 illustrates how the tool for making a subcutaneous pocket shown in FIG. 6 is inserted below the skin in an opening before it is inflated according to one embodiment of the present disclosure.

FIG. 8 illustrates how the tool for making a subcutaneous pocket shown in FIG. 6 is then inflated below the skin in a subsequent step according to an embodiment of the present disclosure.

FIG. 9 illustrates the step of inserting an implant into the pocket created after the removal of the tool for making a subcutaneous pocket shown in FIG. 6 according to an embodiment of the present disclosure.

FIG. 10 illustrates a kit for making a subcutaneous pocket according to another embodiment of the present disclosure.

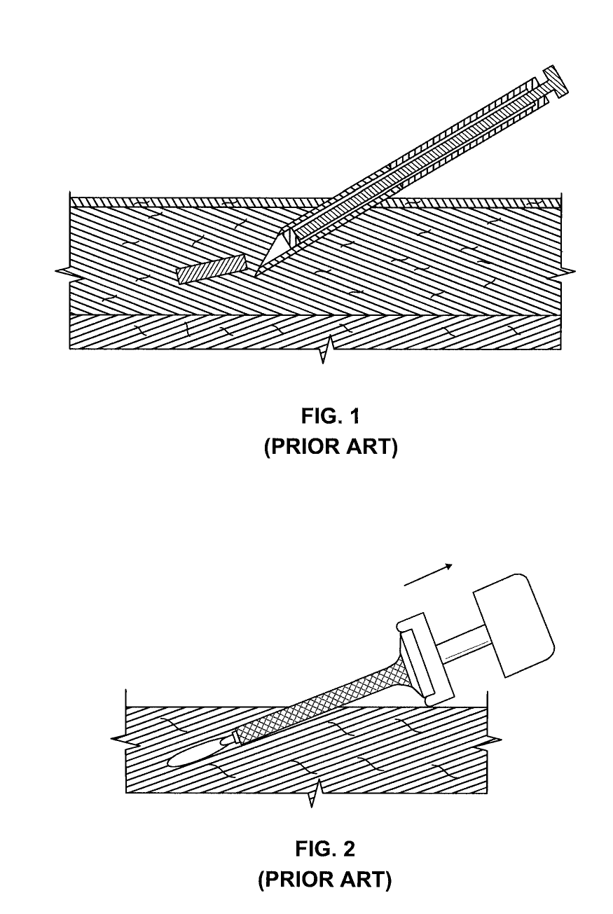

### DETAILED DESCRIPTION OF THE INVENTION

For the purposes of promoting and understanding the principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed and illustrated herein are contemplated as would normally occur to one skilled in the art to which this disclosure relates.

During medical interventions, different devices must be inserted subcutaneously in the human body as part of an interface for external equipment or as internal equipment for autonomous operation of systems within the human body.

In the case of larger inserts, such as, for example, the device described in U.S. Pat. No. 5,041,098, an object must be inserted through an opening in the skin and ultimately rested against a flat surface, such as muscle tissue, or at another precise location. In some cases, pockets or zones must be prepared to receive the device by moving organs and/or removing or displacing layers of fatty tissues. These large objects require a larger opening in the skin and stitches or other means of closure. Tunnels can be created in the fatty tissue located between the skin and the muscle tissue by means of displacement of the fatty tissue either laterally or vertically away from the pocket.

Some of the problems associated with the insertion of large objects include extensive bleeding resulting from rupture of vessels in the tissues, and most importantly, the premature collapse of openings such as tunnels made for insertion of the device. For example, surgeons can cut the skin, make a cut in the fatty tissues, and manually displace part of the tissues to create a zone where the object must be placed. Unless the surgeon maintains constant retraction, the fatty tissue often collapses and the cavity created is partly filled with blood. Current types of operations to insert these large objects require the skills of surgeons and cannot be done by auxiliary staff assisting the surgeon.

Purely for illustrative purposes, the present disclosure is shown to operate with a device of the type described in U.S. Pat. No. 5,041,098, which patent is hereby incorporated herein by reference. The inventor of the present disclosure does not wish to restrict the scope of this invention to this single application. Instead, what is described herein applies with equal utility to any device or object that must be placed below the skin, whatever its configuration and intended use, by simply making small changes to the overall configuration and dimensions of the tool and associated kit.

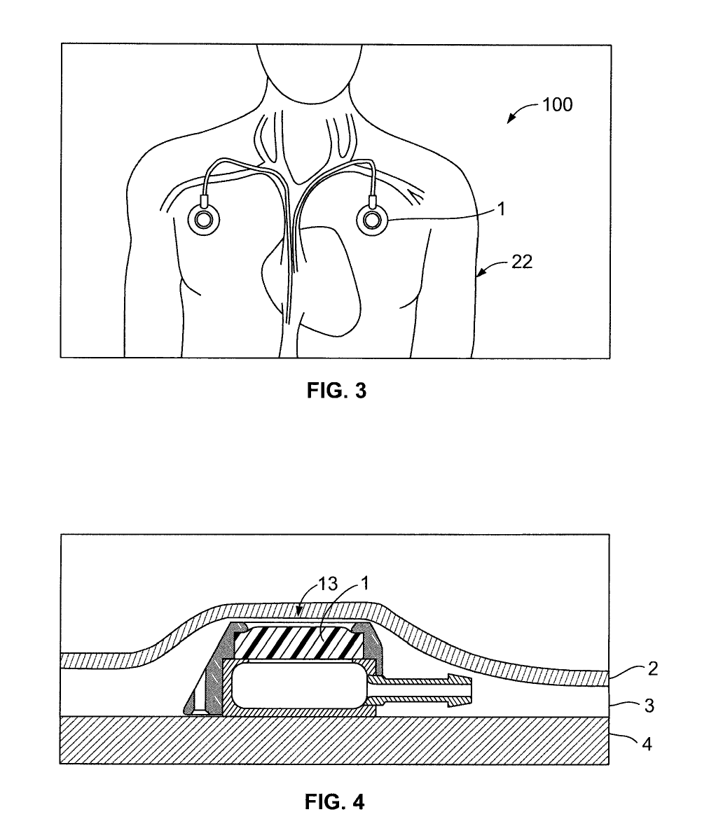

FIG. 3 is taken from U.S. Pat. No. 5,041,098 and shows one possible location 100 where a subcutaneous device can be placed. FIG. 4 is a sectional view of one possible implant as shown in FIG. 3 placed between a layer of muscle tissue 4 and the skin 2 on a human 22 according to an embodiment of the present disclosure. The device 1 is shown resting in a fatty tissue layer 3. While the fatty layer 3 is sandwiched between a first skin layer 2 and a muscle tissue layer 4, those of ordinary skill recognize that what is described here is the judicious placement of the device 1 at a specific location under the skin layer 2 at any position in the body. By way of nonlimiting example, the device 1 can be secured to a cranial bone below the scalp. FIG. 4 shows a possible location of an opening 13 that must be made in the skin layer 2 as a first step of insertion of the device using the tool for making a subcutaneous pocket 37. In one preferred embodiment, the opening 13 made in the skin 2 is roughly the size of a half-dollar coin.

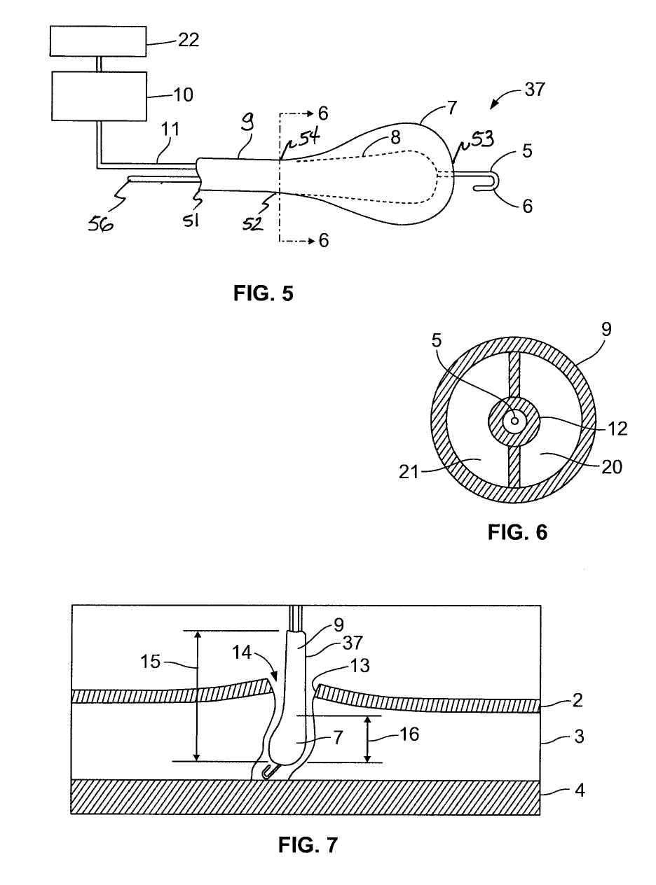

Turning specifically to the tool for making a subcutaneous pocket 37 as illustrated in FIG. 5, a first inflatable volume 9 shown in the embodiment as a cylindrical tube in FIG. 6 includes a proximal end 51 and a distal end 52. The proximal end 51 is connected to at least one pumping means 10 capable of inflating the tool 37 if the tool includes only a single inflatable volume 9 that acts to define both a tunnel and a pocket. FIG. 5 shows how two pumping means 10, 22 can each be connected to different chambers 20, 21 via tubes 56, 11 to fill the first inflatable volume 9 and the second inflatable volume 7, respectively. A portion of the separating wall 12 allows for the introduction and movement of the guide wire 5. FIG. 5 shows a pre-inflation size 8 of the tool 37 using dashed lines and a post-inflation or an intermediate status of the tool 37.

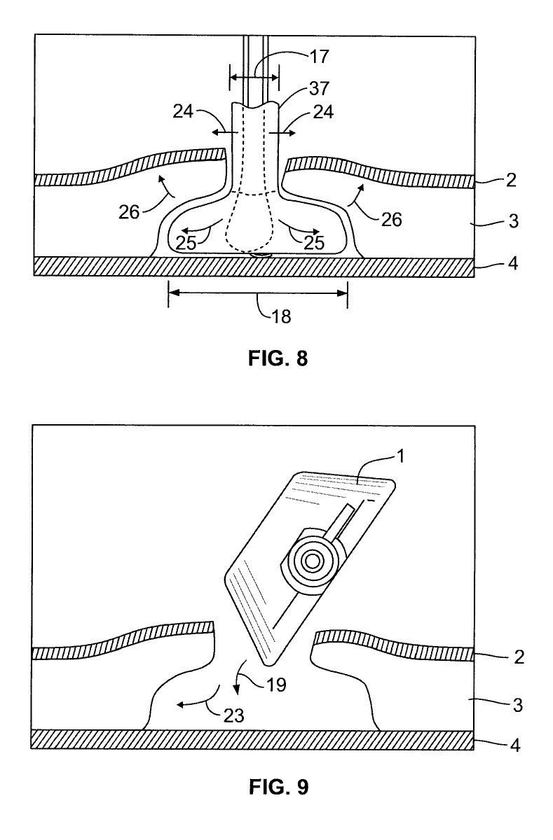

The second inflatable volume 7 includes a first end 54 and a second end 53 in opposition to the first end 54 wherein the first end 54 is connected to the distal end 52 of the first inflatable volume 9. A guide wire 5 is also connected to the proximal end 51 and the second end 53 as shown in one embodiment. This guide wire 5 can be made of any material, including solid or semirigid wire or any other type of wire. In the preferred embodiment, the guide wire 5 is made of stiff wire with a curved end 6 capable of contact and angular deflection when placed in contact with a layer of heavier density. In an alternate embodiment, the guide wire 5 is made of a conductive material such that the tip may act as the pointed tip of a cattery pen where electricity may be used to closed and burn off the tip of small blood vessels. While a rounded tip portion is shown, what is also contemplated is a T shape tip where the flat portion of the T shape can be made to rest against the muscle tissue 4 and can be pushed downwards against the fatty layer 3. In yet another embodiment, the guide wire as shown on FIG. 8 can be bent downwards to allow for the second end 53 of the second inflatable volume 7, but also what is contemplated is a groove made in the second end 53 that allows the tip to enter inwards and let the entire surface of the second end 53 to rest against the muscle tissue 4. In yet another embodiment, the guide wire is a strand with splitting ends that allow for each strand to bend in a different direction when placed against the muscle tissue 4 and allows for the rotation of the tool 37 to displace the fat locally and held with dislodging the superficial fat from the muscle 4.

Again, while one possible configuration of tool for making a subcutaneous pocket 37 is shown, the use of any geometry of inflatable volumes is contemplated. In one contemplated embodiment, the inflatable volumes 7, 9 are made of silicon or rubberized material. While one material is given as the current best mode, the use of any material capable of changing shape or any other type of deformable means is contemplated. For example, if a thermoform material is used, it may be compressed into a small ball and inserted as the inflatable volumes 7, 9 into the body of a patient for expansion in place. In yet another embodiment, the inflatable volumes can be inflated with air, liquid, or simply be filled with a foam capable of expansion under internal or external stimuli. The pumping means 10, 22 can be a thermal source or an electrical source capable of allowing the thermoform material to change shape into the form of a pocket.

Turning to FIGS. 7-9, what is shown is how the tool 37 can be used to create a pocket for the introduction of a device 1. The method may include the creation of a hole in the skin 13 the use of a tool 37 at a predefined length 15 for introduction into the opening 14 until the guide 5 with a curved end or blunted end 6 touches the wall, such as a structural or a preferred layer to feel the stop point, making the wire rest as shown in FIG. 7. Once in place, the first inflatable volume is vertically positioned, the second inflatable volume rests against a muscle tissue as shown in FIG. 8, and the second inflatable volume 7 is inflated as shown by the arrows 25. The fatty tissues retract under the pressure created as shown by the arrows 24. The resulting pocket may be made at a height 16 and a width 18 to accommodate the device as shown in FIG. 8. In a subsequent step illustrated by the arrows 24, the first inflatable volume 9 is inflated to create an opening after the second volume has been inflated 25 and the fat has been moved 26 to create a pocket. One of the advantages of having a two-step inflation system is to prevent bleeding through the skin at 13 because the opening is reduced.

In yet another subsequent step shown as FIG. 9, the first and second inflatable volumes 7, 9 are deflated and the device 1 is slid into the pocket 19 in position 23 after the tool 37 is removed from the pocket. The device 1 is inserted and the area is ready for surgical closure using ordinary closure means.

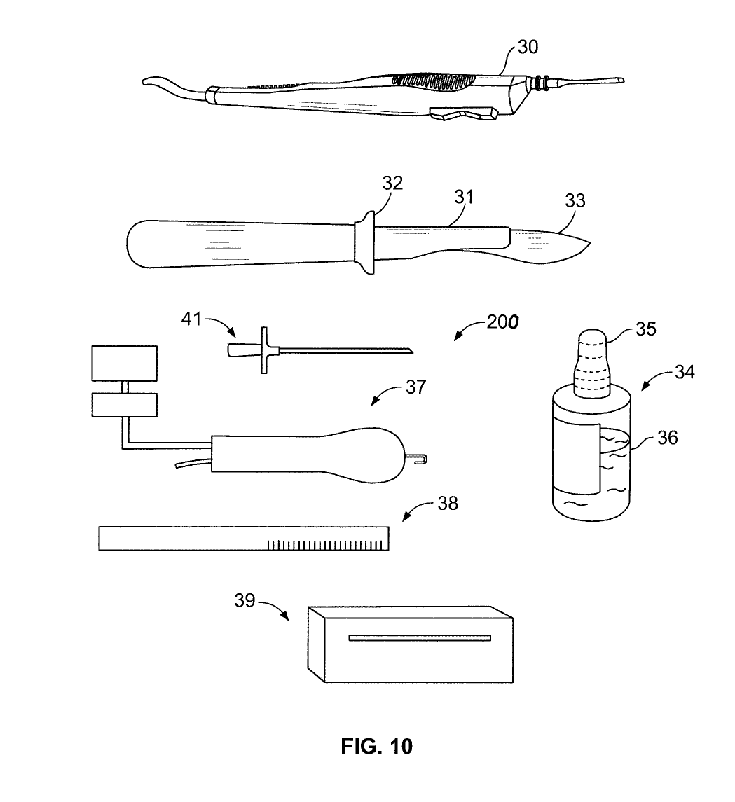

As shown in FIG. 10, the tool 37 can be included as part of a kit 200 for making a subcutaneous pocket. The kit 200 can include a small ruler 38 for measuring the different lengths to be determined during the method of creating a pocket. The kit 200 can also include a cutting device with either a handheld or fixed blade 39 for removing layers or portions of the fatty tissues 3. What is also contemplated is the use of a small bottle 34 with a threaded top having different openings in a nozzle 35 measuring 2 to 3 cm to help deliver a clotting drug such as Thrombin 36 into the opened area. The kit 200 can also include a cattery pen 30 to control blood loss and a specially designed knife 31 with a triangular or regularly shaped blade 33 with a special system 32 to control the distance of insertion of the blade into the patient. The kit 200 can further include a micro introducer 41 with thin wire or guide line or any other of micro introducer 41. The kit 200 is designed to allow nonsurgeons to perform some or part of the different steps of the method for creating a subcutaneous pocket.

What is described is a subcutaneous pocket-making tool 37 with at least a pumping means 10, at least a tube 56 connecting the pumping means 10 to a device 37 including a first inflatable volume 9 and a second inflatable volume 7, where the first inflatable volume 9 in an inflated configuration closes an opening as shown in FIG. 8 in a skin 2 to control bleeding, and where the second inflatable volume 7 in an inflated configuration displaces a layer of fatty tissue to create a subcutaneous pocket also shown in FIG. 8.

The first inflatable volume 9 includes a proximal end 51 and a distal end 52 in opposition, and the second inflatable volume 7 includes a first end 54 and a second end 53 in opposition, and wherein the distal end 52 is in contact with the second end 53 to form a continuously shaped exterior surface as shown in FIGS. 7 and 8. The first and second inflatable volumes 7, 9 includes chambers 20, 21 connected to the at least one tube 56, 11.

In another embodiment, the subcutaneous pocket-making tool 37 includes two pumping means 10, 22 and two tubes 11, 56, each tube 11, 56 connected each to one of the chambers 20, 21 of a first inflatable volume 9 with a proximal end 51 and a distal end 52 in opposition. In FIG. 10, a kit for making a subcutaneous pocket 200 includes a subcutaneous pocket-making tool 37 and at least one of a cattery pen 30 for closing small veins or arteries to limit bleeding into the area of interest, a specially designed knife 31 with for example a moveable stop that can be placed at a fixed and predetermined distance from the cutting edge of the knife, Thrombin 36 as an anti-coagulant, a ruler 38, a cutting device 39, and a micro introducer 41.

In another embodiment, a method for making a subcutaneous pocket is contemplated with the steps of creating an opening 14 in skin 2 at a predefined length with a tool, such as, for example, the knife 31, introducing in the opening 14 a subcutaneous pocket-making tool 37 with a guide 5 until the guide reaches a preferred layer 4, such as a muscle layer, holding the tool in place as shown in FIG. 7 and inflating the first inflatable volume as shown by the arrows 24 in FIG. 8 until the opening 14 is partly closed, and inflating the second volume as shown by the arrows 25 in FIG. 8 until the layer of fatty tissue is displaced 26 creating a subcutaneous pocket as shown in FIG. 9. In yet another step of the method, the volumes 7, 9 are deflated in either order and the tool 37 is removed from the opening. Finally, a device 1 is inserted into the subcutaneous pocket as shown by the arrows 19, 23 in FIG. 9 before the opening 14 is closed using suture or other means for closing skin known in the art.

Finally, what is also contemplated is a method for making a subcutaneous pocket using a kit 200 with the steps of creating an opening 14 in a skin 2 at a predefined length with a tool 31 using a cutting device 31, introducing in the opening 14 a subcutaneous pocket-making tool 37 with a guide 5 until the guide reaches a preferred layer 4, holding the tool 37 in place and inflating the first inflatable volume 9 as shown in FIG. 8 until the opening is partly closed, and inflating the second volume 7 until the layer of fatty tissue is displaced as shown by arrows 26 in FIG. 8 to create a subcutaneous pocket. What is also contemplated is the use of a small ruler 38 in the opening 14 to measure the desired thickness of the subcutaneous pocket, using a special system 32 on a specially designed knife 31 to make an incision to create an opening down to the desired thickness as shown, deflating the second volume 7, deflating the first volume 9, and removing the tool 37 from the opening 14.

It is understood that the preceding is merely a detailed description of some examples and embodiments of the present invention and that numerous changes to the disclosed embodiments can be made in accordance with the disclosure made herein without departing from the spirit or scope of the invention. The preceding description, therefore, is not meant to limit the scope of the invention but to provide sufficient disclosure to one of ordinary skill in the art to practice the invention without undue burden.

What is claimed is:

1. A subcutaneous pocket-making tool, comprising:
at least a pumping means;
at least a tube connecting the pumping means to a device including a first inflatable volume and a second inflatable volume,
wherein the first inflatable volume is an inflated configuration closes an opening in a skin to control bleeding, and wherein the second inflatable volume in an inflated configuration displaces a layer of fatty tissue to create a subcutaneous pocket.

2. The subcutaneous pocket-making tool of claim 1, further comprising a guide wire in the first and second inflatable volumes.

3. The subcutaneous pocket-making tool of claim 2, wherein the guide wire includes a curved end.

4. The subcutaneous pocket-making tool of claim 1, wherein the first inflatable volume includes a proximal end and a distal end in opposition, and the second inflatable volume includes a first end and a second end in opposition, and wherein the distal end is in contact with the second end to form a continuously shaped exterior surface.

5. The subcutaneous pocket-making tool of claim 1, wherein each of the first and second inflatable volumes includes a chamber connected to the at least one tube.

6. The subcutaneous pocket-making tool of claim 1, wherein the pumping means includes an electrical source or a thermal source.

7. The subcutaneous pocket-making tool of claim 1, wherein the first inflatable volume and the second inflatable volume are made of silicon or rubberized material.

8. The subcutaneous pocket-making tool of claim 1, wherein the second inflatable volume is inflated with a medium selected from a group consisting of air, water, or foam.

9. A subcutaneous pocket-making tool, comprising:
two pumping means;
two tubes, each connected to one of the pumping means and to a chamber of a first inflatable volume with a proximal end and a distal end in opposition, and connected to a second inflatable volume with a first end and a second end in opposition, wherein the distal end is in contact with the second end to form a continuously shaped exterior surface, and
a guide wire with a curved end in the first and second inflatable volumes,
wherein the first inflatable volume is an inflated configuration closes an opening in a skin to control bleeding, and wherein the second inflatable volume in an inflated configuration displaces a layer of fatty tissue to create a subcutaneous pocket.

10. A kit for making a subcutaneous pocket for a subcutaneous device, the kit comprising:
a subcutaneous pocket-making tool with at least a pumping means, at least a tube connecting the pumping means to a device including a first inflatable volume and a second inflatable volume, and wherein the first inflatable volume is an inflated configuration closes an opening in skin to control bleeding, and wherein the second inflatable volume in an inflated configuration displaces a layer of fatty tissue to create a subcutaneous pocket; and
at least one of a group consisting of a cattery pen, a specially designed knife, Thrombin, a ruler, a cutting device, and a micro introducer.

11. The kit for making a subcutaneous pocket for a subcutaneous device of claim 10, further comprising at least two from the group consisting of a cattery pen, a specially designed knife, Thrombin, a ruler, a cutting device, and a micro introducer.

12. The kit for making a subcutaneous pocket for a subcutaneous device of claim 10, further comprising at least three from the group consisting of a cattery pen, a specially designed knife, Thrombin, a ruler, a cutting device, and a micro introducer.

13. A method for making a subcutaneous pocket, the method comprising the steps of:
creating an opening in skin at a predefined length with a tool;
introducing in the opening a subcutaneous pocket-making tool with a guide until the guide reaches a preferred layer, the tool having at least a pumping means, at least a tube connecting the pumping means to a device including a first inflatable volume and a second inflatable volume;
holding the tool in place and inflating the first inflatable volume until the opening is partly closed; and
inflating the second volume until the layer of fatty tissue is displaced creating a subcutaneous pocket.

14. The method for making a subcutaneous pocket of claim 13, further comprising the steps of deflating the second volume, deflating the first volume, and removing the tool from the opening.

15. The method for making a subcutaneous pocket of claim 14, further comprising the step of inserting a device into the subcutaneous pocket and closing the opening using a means of closure.

16. A method for making a subcutaneous pocket using a kit for making a subcutaneous pocket, the method comprising the steps of:
creating an opening in a skin at a predefined length with a tool using a cutting device;
introducing in the opening a subcutaneous pocket-making tool with a guide until the guide reaches a preferred layer, the tool having at least a pumping means, at least a tube connecting the pumping means to a device including a first inflatable volume and a second inflatable volume;
holding the tool in place and inflating the first inflatable volume until the opening is partly closed; and
inflating the second volume until the layer of fatty tissue is displaced creating a subcutaneous pocket.

17. The method of claim 16, further comprising the steps of placing a small ruler in the opening to measure the desired thickness of the subcutaneous pocket, using a special system on a specially designed knife to make an incision to create an opening down to the desired thickness.

18. The method for making a subcutaneous pocket of claim 17, further comprising the steps of deflating the second volume, deflating the first volume, and removing the tool from the opening.

19. The method for making a subcutaneous pocket of claim 18, further comprising the step of inserting a device into the subcutaneous pocket and closing the opening using a means of closure.

20. The method of claim 17, further comprising the step of using an anticoagulant.

21. The method of claim 17, further comprising the steps of using a cattery pen to cauterize bleeding vessels in the opening.
